(19) United States
(12) Patent Application Publication
(10) Pub. No.: US 2006/0002866 A1
(43) Pub. Date: Jan. 5, 2006

(54) Method for protecting the skin from aging

(76) Inventors: Pauly Gilles, Nancy (FR); Jean-Luc Contet-Audonneau, Saint-Max (FR); Louis Danoux, Saulxures les Nancy (FR); Christine Jeanmaire, Nancy (FR); Yanusz Wegrowski, Bezannes (FR); Francois-Xavier Maquart, Reims (FR)

Correspondence Address: COGNIS CORPORATION;PATENT DEPARTMENT, 300 BROOKSIDE AVENUE, AMBLER, PA, 19002, US

(21) Appl. No.: 10/530,821

(22) PCT Filed: Sep. 27, 2003
(86) PCT No.: PCT/EP03/10767

§ 371 Date: Apr. 8, 2005

(30) Foreign Application Priority Data
Oct. 8, 2002 (EP) 02292475.7

(51) Int. Cl. A61K 7/42; A61K 7/06

(52) U.S. Cl. 424/59; 424/70.13

(57) ABSTRACT

The present invention relates to a cosmetic treatment method for improving and/or protecting the human skin, scalp and/or mucous membrane, characterized in that a preparation containing at least one substance which modulates proteoglycans, more particularly lumican and/or syndecan and/or versican and/or decorin and/or glypican and/or biglycan, is topically applied. Such substances include plant extracts of Pisum sativum and Vigna aconitifolia, mannitol, cyclodextrin, yeast extract, disodium succinate, phytosterols, triterpenes, triterpene saponins, TGFβ, IL4, flavonoids, and flavonoid derivatives. The method can be applied for protecting the skin from aging and other detrimental influences of environmental pollutants and UV radiation, for boosting the functions of the dermal/epidermal junctions, and for improving wound healing.

### FIELD OF THE INVENTION

This invention relates generally to cosmetic preparations and, more particularly, to a cosmetic process for protecting the human skin, scalp and/or mucous membrane against ageing, oxidative stress and the harmful effects of environmental toxins and UV radiation. The present invention also relates to the use of a substance which modulates lumican and/or syndecan and/or versican and/or decorin and/or glypican and/or biglycan for the production of cosmetic preparations and preparations for treating particular skin diseases.

### PRIOR ART

The extracellular matrix of the connective tissue consists of a plurality of macromolecules which form complex three-dimensional networks, interact with one another and with the cells of the connective tissue and thus contribute significantly to the structural integrity of the tissue. However, not only do they form a link between individual cells and tissues, they also provide for the regulation of cell growth, cell supply and cell differentiation through a filter and transport function (of growth factors, inhibitors or hormones) and through special receptor bonds. Key constituents of the extracellular matrix include collagen, elastin, glycoproteins, hyaluronic acid, glycosaminoglycans, proteoglycans and glycoproteins which belong to the non-fibrous basic structure.

Proteoglycans are macromolecules with a central protein to which one or more glucosaminoglycan side chains are covalently bonded. They represent the main component in the extracellular matrix and comprise a number of different molecules which may be roughly divided into large and small proteoglycans. Proteoglycans can be found at all levels of the basal membrane which forms a largely homogeneous layer immediately below the basal epithelial cells and which can be divided into three layers. The uppermost part, which directly adjoins the cells, is the Lamina lucida which is followed by the Lamina densa as the middle layer of the basal membrane and then by the outer Lamina fibroreticularis.

In the basal membrane, proteoglycans contribute towards tissue strength via the bonds to fibrous tissue constituents and, by binding water, influence the elasticity of the skin. Studies have shown that the increased wrinkling of the skin and the reduction in elasticity is associated with collagen fibers.

During ageing, the skin undergoes major changes in its mechanical properties, in its ability to retain water and in its firmness and elasticity. Proteoglycans have a major influence on these changes. In addition, in normal ageing processes, an opposite change is observed, i.e. a reduction in the large chondroitin sulfate proteoglycans (versican) and a parallel increase in the numbers of small dermatan sulfate proteoglycans (decorin) with increasing age.

It is known that disease-induced changes in the skin can be influenced by changes in the proteoglycans. Thus, International patent application WO 01/17560 describes the treatment and prevention of bacterial infections using substances which inhibit the release of syndecan-1. International patent application WO 94/12162 discloses the reduction in tumour growth and the stimulation of hair growth by the stimulation of syndecan. In addition, it has been shown that scar formation is reduced or even prevented by administration of decorin, biglycan and fibromodulin (cf. International patent application WO 93/09800).

In view of the demand for preventing ageing-induced changes in the skin and for effective protection of the skin against environment-induced ageing effects, the problem addressed by the present invention was to provide new mechanisms for improving the skin, scalp and/or mucous membrane which would contribute to a delay in skin ageing and to protection of the skin, scalp and/or mucous membrane against environmental influences, oxidative stress, toxic substances or UV radiation and which could therefore be effectively used in cosmetic and dermopharmaceutical preparations for topical application.

### DESCRIPTION OF THE INVENTION

The present invention relates to a cosmetic treatment process for improving and/or protecting the human skin, scalp and/or mucous membrane, characterized in that a preparation containing at least one substance which modulates proteoglycans, more particularly lumican and/or syndecan and/or versican and/or decorin and/or glypican and/or biglycan, is topically applied.

The present invention also relates to the use of a substance which modulates lumican and/or syndecan and/or versican and/or decorin and/or glypican and/or biglycan for the production of cosmetic preparations for protecting the human skin, scalp and/or mucous membrane against ageing, for the production of cosmetic preparations for protection against oxidative stress, for protection against toxic environmental influences, for protection against damage by UV light and for improving the functions of the dermal/epidermal junctions and to their use for the production of dermopharmaceutical preparations for improving the healing of wounds and preparations for treating alopecia, cellulitis or roseacea.

It has surprisingly been found that the modulation of proteoglycans, more particularly lumican, syndecan, versican, decorin, glypican and/or biglycan, leads to an improvement in, and to protection of, the human skin, scalp or mucous membrane. Thus, firming of the skin, increased elasticity and a better water-binding capacity have been observed, even after exposure to UV radiation. The capacity of the skin for regeneration is distinctly improved, so that renewal of the skin and particularly the healing of wounds take place far more quickly which also affords advantages in the treatment of inflammatory skin diseases, more particularly alopecia, cellulitis and roseacea.

Substances which modulate proteoglycans, more particularly lumican, syndecan, versican, decorin, glypican and/or biglycan, may be used on their own or in combination with other active principles which
strengthen dermal macromolecules and make them more resistant to non-enzymatic glycosylation and thus protect the skin against toxic environmental poisons and oxidative stress,
maintain the balance of the growth factors in aged human skin in order to improve the renewal and repair of the skin after damage by UV radiation or in wound healing processes,
support the formation of microfibrils in the human skin and hence provide protection against manifestations of skin ageing,
improve the functions of the dermal/epidermal junctions (DEJ) through improved anchorage by strengthening of the microfibrils,
increase the water-binding capacity of the skin and thus contribute to firmer skin,
reduce the formation of microwrinkles and reduce further wrinkling,
delay the appearance of alopecia,
reduce skin changes attributable to cellulitis and roseacea,
reduce the development of inflammatory processes which lead to irritation, reddening and itching
influence the synthesis of melanin in the skin,
strengthen the immune system of the skin and thus improve the defence system against harmful environmental influences.

Modulators which have proved to be suitable are plant extracts, more particularly the extract of Pisum sativum and/or Vigna aconitifolia, extracts of microorganisms and/or fermentation products of vegetable origin. However, lumican, syndecan, versican, decorin, glypican and/or biglycan can also be modulated by the application of at least one substance selected from the group consisting of mannitol, cyclodextrin, yeast extract and disodium succinate. The combination of these constituents in particular leads to an advantageous effect.

Modulation of the molecules by at least one substance selected from the group consisting of
phytosterols such as, for example β-sitosterol, campesterol, brassicasterol, Δ5-avennasterol, α-spinasterol or stigmasterol;
phytoestrogens, such as isoflavones (genistein, daidzein), stilbenes, lignan;
triterpenes, such as lupeol, ursolic acid, arjunolic acid, oleanolic acid;
triterpene saponins and steroid saponins, such as sapogenin, diosgenin, hecogenin, smilagenin, sarsapogenin, tigogenin, yamogenin, yuccagenin and bassic acid;
peptides, more particularly those which correspond to the growth factors TGFβ, IL4 and
flavonoids and flavonoid derivatives.

Besides these substances or plant extracts, the cosmetic preparations may also contain UV protection factors and/or antioxidants. The combination of substances which modulate lumican and/or syndecan and/or versican and/or decorin and/or glypican and/or biglycan with UV protection factors and/or antioxidants leads through the various mechanisms to a synergistic mode of action and affords excellent protection against harmful effects and ageing of the skin by UV light.

Lumican

Lumican belongs to the family of keratan sulfates, a group of leucine-rich proteoglycans (LRP), which are located in the skin together with fibrillar collagen. In the fibrils of various connective tissues, lumican contributes to the elasticity and stability of the tissue. Histologically, collagen strands appear disorganized and without strength in lumican-poor tissue. In the subcutaneous tissue in particular, there is a lack of order and orientation of collagen strands and fibroblasts. The histological anomalies can also be verified with an electron microscope by examining the organization of the collagen matrix, enlarged interfibrillar spaces and modified morphology of the fibrils.

Syndecan

Syndecans belong to the group of transmembranal heparan sulfate proteoglycans which act as co-receptors with integrin and growth factor tyrosinekinase receptors. Through their negative surface charge, they bind to positively charged matrix sections and form firmly anchored contacts in the basal lamina. Syndecan-4 is an important surface receptor for wound healing and angiogenesis. Syndecan-1 is the main heparan sulfate proteoglycan of the epidermis. It contains heparan sulfate and chondroitin sulfate and is found in mature, fully developed tissue, mainly in simple and layer-form epithelia. In the epidermis, syndecan-1 is found in particular in the suprabasal cell layer, Lamina rara. It has a significant share in the migration and proliferation of keratinocytes in wound healing. Scientists have recently found an enhancer in syndecan-1 (FiRE—fibroblast growth factor-inducible response element—in the promoter of the syndecan-1 gene) which activates gene expression in keratinocytes at the edges of wounds. They were able to show that the composition of the extracellular matrix and the availability of growth factors are influenced by the epidermal regulation of syndecan-1 expression, so that FiRE is a new goal for the gene regulation of the extracellular matrix.

Versican

Versican is one of the large chondroitin sulfate proteoglycans which are found in the Lamina densa, the middle part of the basal membrane, and in the Lamina fibroreticularis.

Decorin

Decorin is one of the smallest proteoglycans of the leucine-rich family (LRP). A dermatan sulfate proteoglycan which is largely found in the extracellular matrix of collagen-rich tissue where it covers the surface of collagen fibrils.

Glypican

Glypican is a proteoglycan which is anchored by linkage to membrane through glycosyl phosphatidyl inositol and which binds antithrombin and lipoprotein lipases on the endothelial cell surface.

Biglycan

Biglycan is a small proteoglycan which is released through mesenchymal cells. It is also synthesized by endothelial and epithelial cells and is found mainly in the pericellular gap, but also in the cell core. Some factors, such as TGFβ and b-FGF, regulate biglycan synthesis by fibroblasts. The function of biglycan is still largely unknown.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzo-phenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol 1789) or 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4-methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

### EXAMPLES

Glucosaminoglycans and proteoglycans of human skin varying in age were investigated to show that ageing-induced changes of proteoglycans contribute to the corresponding appearance of the skin and to its mechanical properties. To this end, samples of child's skin, adult's skin and aged skin were taken and cell cultures of keratinocytes and fibroblasts were cultivated from them.

In order to determine the features of human skin as a function of age, the following classical techniques were used [19-WEGROWSKI, Y.; PALTOT, V.; GILLERY, P.; KALIS, B.; RANDOUX, A.; MAQUART, F. X.; Biochemical Journal 2995, 307, 3, 673-678; WEGROWSKI, Y.; GILLERY, P.; KOTLARZ, G.; PERREAU, C.; GEORGES, N. and MAQUART, F. X.; Molecular and Cellular Biochemistry, 200, 205, 125-131].

For glucosaminoglycans:

Use of radioactively marked molecules, such as tritium-marked glucosamines, for all glucosaminoglycans except keratan sulfate and ³⁵S for all sulfated glucosaminoglycans. Quantitative determination of the marked molecules in glucosaminoglycans and electrophoresis techniques.

For proteoglycans:

Northern Blot technique for determining the RNA messengers in cells of low molecular weight proteoglycans, such as lumican and syndecan.

1. Investigation of Human Dermal Fibroblasts from Donors in Different Age Categories

The quantity of RNA messengers of the proteoglycan lumican was determined in a fibroblast culture by the Northern Blot technique. The results are set out in Table 1 as the ratio of the quantity of RNA messengers for lumican to the quantity of ¹⁸S ribosomal RNA.

TABLE 1
Ratio of the quantity of RNA messengers for lumican to the
quantity of ¹⁸S ribosomal RNA for different age categories

| Age | No. of | Mean value of |
|---|---|---|
| (years) | volunteers | [lumican RNAm]:[18S ribosomal RNA] ratio |
| 1-15 | 8 | 4.0 |
| 16-50 | 8 | 2.7 |
| 51-71 | 8 | 1.9 |

The data in Table 1 clearly show that the quantity of RNA messengers for lumican decreases with increasing age of the fibroblast donor. This can mean that the lumican synthesis rate in the skin cells has a significant influence on the appearance of ageing skin.

2. Investigation of Human Keratinocytes from Donors in Different Age Categories

The quantity of RNA messengers of the proteoglycan syndecan-1 was determined in a keratinocyte culture by the Northern Blot technique. The results are set out in Table 2 as the ratio of the quantity of RNA messengers for syndecan-1 to the quantity of ¹⁸S ribosomal RNA.

TABLE 2
Ratio of the quantity of RNA messengers for syndecan-1 to the
quantity of ¹⁸S ribosomal RNA for different age categories

| Age | No. of | Mean value of |
|---|---|---|
| (years) | volunteers | [syndecan-1 RNAm]:[18S ribosomal RNA] ratio |
| 1-15 | 4 | 5.1 |
| 16-50 | 4 | 3.2 |
| 51-71 | 4 | 3.1 |

Even comparable investigation of the proteoglycan syndecan-1 shows a reduction in the RNA messengers with increasing age of the keratinocyte donors, so that it must be assumed that there is a reduction in the synthesis of this proteoglycan. This is also an indication that the synthesis rate of the proteoglycans can have a significant influence on the ageing of skin.

3. Stimulation of the Lumican mRNA Content in MRC5 Fibroblasts by IGF-1.

The stimulation of the lumican mRNA content in MRC5 fibroblasts incubated with increasing concentrations of the growth factor IGF-1 (Insulin-like Growth Factor 1, Sigma-Aldrich) was investigated. The content of mRNA of the proteoglycan lumican in cultures of human fibroblasts—MRC5 fibroblasts—was determined by the Northern Blot method.

TABLE 3
Results as lumican mRNA content against housekeeping gene
36B4 mRNA as a function of the IGF-1 concentration

| | Mean value |
|---|---|
| Concentration of IGF-1 (ng/ml) | [mRNA lumican/36B4 mRNA] |
| 0 | 0.86 |
| 0.1 | 1.38 |
| 1 | 2 |
| 10 | 3.2 |

The results clearly show that IGF-1 in cultures of MRC5 fibroblasts increases the mRNA content for lumican.

1-17. (canceled)

18. A method of protecting the human skin against aging, said method comprising applying to the skin a composition containing at least one substance which modulates at least one proteoglycan selected from a group consisting of lumican, syndecan, versican, decorin, glypican and biglycan.

19. A method according to claim 18 wherein the at least one substance modulates lumican, syndecan or decorin.

20. A method according to claim 19 wherein the at least one substance is selected from at least one plant extract.

21. A method according to claim 20 wherein the at least one plant extract is selected from an extract of Pisum sativum and Vigna aconitifolia.

22. A method according to claim 18 wherein the at least one substance is selected from a group consisting of mannitol, cyclodextrin, yeast extract and disodium succinate.

23. A method according to claim 18 wherein the at least one substance is selected from a group consisting of phytosterols, triterpenes, triterpene saponins, TGFβ, IL4, flavonoids, and flavonoid derivatives.

24. A method of improving the regeneration and healing of the human skin, said method comprising applying to the skin a composition containing at least one substance which modulates at least one proteoglycan selected from a group consisting of lumican, syndecan, versican, decorin, glypican and biglycan.

25. A method according to claim 24 wherein the at least one substance modulates lumican, syndecan or biglycan.

26. A method according to claim 24 wherein the at least one substance is selected from at least one plant extract.

27. A method according to claim 26 wherein the at least one plant extract is selected from an extract of Pisum sativum and Vigna aconitifolia.

28. A method according to claim 24 wherein the at least one substance is selected from a group consisting of mannitol, cyclodextrin, yeast extract and disodium succinate.

29. A method according to claim 24 wherein the at least one substance is selected from a group consisting of phytosterols, triterpenes, triterpene saponins, TGFβ, IL4, b-FGF, flavonoids, and flavonoid derivatives.

30. A method of increasing the mRNA content for lumican, said method comprising incubating MRC5 fibroblasts with IGF-1.
